(19) United States
(12) Patent Application Publication
(10) Pub. No.: US 2025/0194778 A1
(43) Pub. Date: Jun. 19, 2025

(54) REMOVABLE, INFLATABLE BAG ADAPTED FOR INSERTION INTO A BACKPACK

(71) Applicant: WC DEVELOPMENT LLC, Missoula, MT (US)

(72) Inventors: Walter S. Hailes, Missoula, MT (US); Carson J. Wilde, Missoula, MT (US)

(73) Assignee: WC DEVELOPMENT LLC, Missoula, MT (US)

(21) Appl. No.: 18/979,791

(22) Filed: Dec. 13, 2024

Related U.S. Application Data
(60) Provisional application No. 63/609,919, filed on Dec. 14, 2023.

(51) Int. Cl. A45F 3/04; A45F 3/00

(52) U.S. Cl. CPC A45F 3/04; A45F 2003/003

(57) ABSTRACT

A removable, inflatable bag for insertion into a backpack is described. The inflatable bag includes a body having an interior cavity for storing weighted items therein, and an inflation valve unit arranged on the body and adapted to receive pressurized air therethrough to fully inflate the inflatable bag, such that internal surfaces within the interior cavity expand inward, and external surfaces of the body expand outward to fill the volume within the backpack. With the inflatable bag arranged within the backpack and fully inflated, and with the weighted items contained within the interior cavity and the inflatable bag fully inflated and closed, the surfaces of the interior cavity bear against the weighted items to prevent any movement thereof, eliminating any shifting of the weighted items during a wearer's movement activities with the backpack.

### CROSS-REFERENCE TO RELATED APPLICATION

The present application claims priority under 35 U.S.C 119 (e) to co-pending U.S. Provisional Patent Application Ser. No. 63/609,619 to the co-inventors, filed Dec. 13, 2023, the entire contents of which is hereby incorporated by reference herein.

### BACKGROUND

### 1. Field

The example embodiment in general relates to a removable, inflatable bag for insertion into a backpack, more specially to a bag that when filled with weighted items and with its interior volume fully inflated, is then placed within a primary interior storage compartment of the backpack such that it fills the volume of that storage compartment, thereby eliminating any shifting of movement of the weighted items therein as a wearer of the backpack moves along conducting movement activities such as hiking, climbing, walking and the like.

### 2. Related Art

The best way to train for a big outdoor adventure, whether it's climbing a mountain, hiking a trail, or ski touring in deep powder is by carrying a weighted backpack. As such, for avid hikers and climbers (or training military personnel), it is often desirable to train with weighted backpacks or rucksacks prior to commencing a challenging ascent or hike, to build up strength and stamina. FIG. 1 is a rear plan view of a conventional backpack 50 with external compression straps 55. When using a rucksack or a backpack 50 such as shown in FIG. 1 for training activities, such as moving/hiking up a hill or mountain, the climber or hiker typically wants to put weight into the primary interior storage compartment within the backpack 50 for this training. They might start with 25-35 lbs. of weight and gradually increase the weight over subsequent practice hikes, up to 60 lbs. or even more depending on the intensity of training.

Although these backpacks 50 are made to carry such big and heavy loads, these big and bulky weighted loads can be hard to load, unload, and store securely (without movement) within the backpack 50. These steel plates, dumbbells, and water bottles are thus too dense for the wearer of the backpack 50 to carry the load correctly. Also, a problem then typically arises, where the backpack 50 becomes limp, the added loose weight skinks to the bottom of the interior storage compartment. The movable and dense weight loads, as they sit at the bottom of the backpack 50, tend to painfully dig into the wearer's lower back as they walk, or flop from side to side within backpack 50, making the backpack 50 feel unbalanced and potentially leading to injury.

Thus, the weight inserted into the interior storage compartment is moving around within the bottom of the backpack on a hike, its weight unevenly distributed. Even if the hiker adds towels into the interior storage compartment around the loose weight, the towels get compressed and shift around. So the moving weight problem is not eliminated by the use of towels.

Accordingly, the primary interior storage compartment within the backpack 50 essentially needs to be completely full to eliminate weight shifting therein on wearer movement with the backpack 50. Otherwise, and as time goes on during a practice hike for example, the weight shifting can turn into a larger problem for the wearer, often leading to strains and enhanced back pain as noted above. What is needed is a means or mechanism that when employed can ensure that a backpack's interior storage volume is completely full so as to eliminate movement of any weighted items therein during a hike or ascent.

### SUMMARY

An example embodiment of the present invention is directed to a removable, inflatable bag for insertion into an interior storage compartment of a backpack. The inflatable bag includes a body having an interior cavity for storing one or more loose, weighted items through a re-closeable opening at a top end thereof. The inflatable bag also includes an inflation valve unit arranged on an external surface of the body and adapted to receive pressurized air therethrough to fully inflate the body, such that, during inflation, internal surfaces within the interior cavity of the body expand inward, and external surfaces of the body expand outward to fill the volume of the internal storage compartment of the backpack. With the inflatable bag arranged in the interior storage compartment of the backpack and fully inflated, and with the one or more loose, weighted items contained within the interior cavity of the body and the top end opening of the fully inflated bag closed, the surfaces of the interior cavity bear against the one or more loose, weighted items to prevent any movement thereof, eliminating any shifting of the one or more loose, weighted items during a wearer's movement activities with the backpack.

Another example embodiment is directed to a combination comprising a backpack and an inflatable bag. The backpack has a re-closable opening at a top end thereof and an interior storage compartment having a volume that is accessible through the re-closable opening, and the inflatable bag is adapted for insertion into the interior storage compartment of the backpack via the re-closable opening of the backpack for use in weighted backpack training activities by a wearer of the backpack. The inflatable bag further includes a body having an interior cavity for storing weighted items therein through a re-closeable opening at a top end thereof, and an inflation valve unit adapted arranged on a surface of the body and adapted to receive pressurized air therethrough to fully inflate the body of the inflatable bag, such that, during inflation, internal surfaces within the interior cavity of the body expand inward, and external surfaces of the body expand outward to fill the volume of the internal storage compartment of the backpack. Any weighted items added into the interior cavity of the body with the inflatable bag fully inflated are firmly secured as the surfaces of the interior cavity bear against the weighted items, eliminating any shifting of the weighted items during a wearer's movement activities wearing the weighted backpack, the inflated bag therein filling the volume of the interior storage compartment.

Another example embodiment is direction to a combination comprising an inflatable bag and a storage bag. Namely, the inflatable bag is adapted for insertion into an interior storage compartment of a backpack via a re-closable opening in the backpack for use in weighted backpack training activities by a wearer of the backpack. The inflatable bag has an interior cavity for storing weighted items therein, and has an inflation valve unit arranged thereon that is adapted to receive pressurized air therein. The storage bag is adapted for storing the inflatable bag therein when not in use within the backpack. The storage bag serves a dual function as a bag pump to provide pressurized air into the inflation valve unit to fully inflate the inflatable bag. For any weighted items added into an interior cavity of the inflatable bag when the bag has been fully inflated by the storage bag, the fully inflated bag arranged within the interior storage compartment of the backpack to realize a weighted backpack, the weighted items are firmly secured as inflated surfaces within the interior cavity bear against the weighted items. This eliminates any shifting of the weighted items during a wearer's movement activities with the weighted backpack, the inflatable bag fully inflated filling the volume of the interior storage compartment.

### BRIEF DESCRIPTION OF THE DRAWINGS

The example embodiment will become more fully understood from the detailed description given herein below and the accompanying drawings, wherein like elements are represented by like reference numerals, which are given by way of illustration only and thus are not limitative of the example embodiment herein.

FIG. 1 is a photograph of a rear plan view of a conventional backpack with compression straps that is loaded with loose weight within its major interior storage compartment.

FIG. 2 is a photograph of a perspective view of a removable, inflatable bag showing a top flap thereof closed and fastening strap tightened, the inflatable bag adapted for insertion into the conventional backpack according to the example embodiment.

FIG. 3 is a rendering of a front plan view of the inflatable bag similar to that shown in FIG. 2 but with the fastening strap loosened and top flap flipped open to access an interior cavity thereof.

FIG. 4 is a rendering of a rear plan view of the inflatable bag shown in FIGS. 2 and 3.

FIG. 5 is a rendering of a left-side elevational view of the inflatable bag shown in FIGS. 2 and 3.

FIG. 6 is a partial cutaway rendering of the left-side elevational view to illustrate how interior facing surfaces of the bag, when the bag is being inflated, expand inward to secure a loose weight therein.

FIG. 7 is a photograph of the backpack with top opened and laying on its side with the inflatable bag according to the example embodiment having been inserted therein, with a weight shown for insertion into the cavity of the inflatable bag.

FIG. 8 is a photograph of the inflatable bag with weight therein shown in FIG. 7, with an inflation valve unit of the inflatable bag connected to a corresponding air valve in a storage bag which is used as an air pump for inflation.

FIG. 9 is an enlarged view of the boxed area in FIG. 8 to show more detail around the valve connections between the inflatable bag and its storage.

FIG. 10 is diagram showing example component constructions of the valve units in both the inflatable bag and the storage bag as well as how the connection between the two is made.

FIG. 11 is a rear plan view of the conventional backpack shown in FIGS. 1, 7 and 8 with the fully inflated bag therein.

FIG. 12 is a photographed side view of a person wearing a backpack with a loose 25 pound weight inserted into the bottom of the backpack's primary storage compartment.

FIG. 13 is a photographed side view of the same person shown in FIG. 12, but with the same weight firmly secured within the fully inflated bag inside the backpack's primary storage compartment, according to the example embodiment.

FIG. 14 is a photographed side view of a person wearing a backpack and walking on a treadmill with a loose weight within the backpack's primary storage compartment, so as to show load location of the loose weight and force vectors imparted away from the person.

FIG. 15 is a photographed side view of the same person shown in FIG. 14, but shows a different load location and force vectors imparted toward the person when the same weight is firmly secured within the fully inflated bag inside the backpack's primary storage compartment.

### DETAILED DESCRIPTION

As to be described in detail hereafter, an example embodiment is directed to an inflatable bag for use within a backpack to firmly secure weight therein. An optimal way to train for a big outdoor adventure requiring exertion, whether it's climbing a mountain, hiking a trail, or ski touring in deep powder, is by carrying a weighted backpack. The example inflatable bag within the backpack described hereafter allows the backpack wearer to carry any load in a secure and comfortable way, with the load firmly secured to eliminate shifting thereof, the load distributed so as to minimize undesirable forces generated by a loose backpack with weighted items shifting inside that could lead to discomfort, strains and/or further injury.

The inflatable bag described hereafter is designed to secure loose, moveable weight within the backpack, so as to provide a stable and comfortable way to turn the backpack into the perfect training tool for the wearer thereof. The example bag described hereafter in more detail is packable, inflatable and versatile as a training tool. Once inflated and then inserted within the backpack, the inflatable bag works in tandem with the backpack, expanding both inwards within itself to secure any inserted weight of choice, and outwards into the interior backpack volume.

The inflatable bag has a generally flexible shape that upon inflation may match up with the interior profile of a primary storage compartment of the backpack so as to fill the backpack interior, expanding the backpack. As to be discussed in further detail, the inflatable bag is composed of a body having a flexible and closeable top flap that, when opened, permits access to an interior cavity for placing weighted articles of choice therein, so as to add weight to the backpack.

The inflatable bag includes an inflatable valve unit that can be used by the wearer to blow air directly into a one-way valve to inflate the bag. In one particular example as described hereafter, the inflatable bag when not in use can be stored within a companion storage bag. The storage bag also has an air supply valve that can attached to the inflatable so that the storage bag can be used as an air pump. Additionally, as shown hereafter, a front surface of the inflatable bag (which when installed faces forward to the back of the wearer) includes a centrally located baffle region, or baffle, that will not inflate as the bag is inflated, so as to divert air around either side thereof. When the bag is inserted into the backpack's storage compartment and inflated, the location of the resilient baffle with inflated portions on either side thereof provides support and comfort to the back of the user, generally removing pressure along the mid-spine area of the wearer.

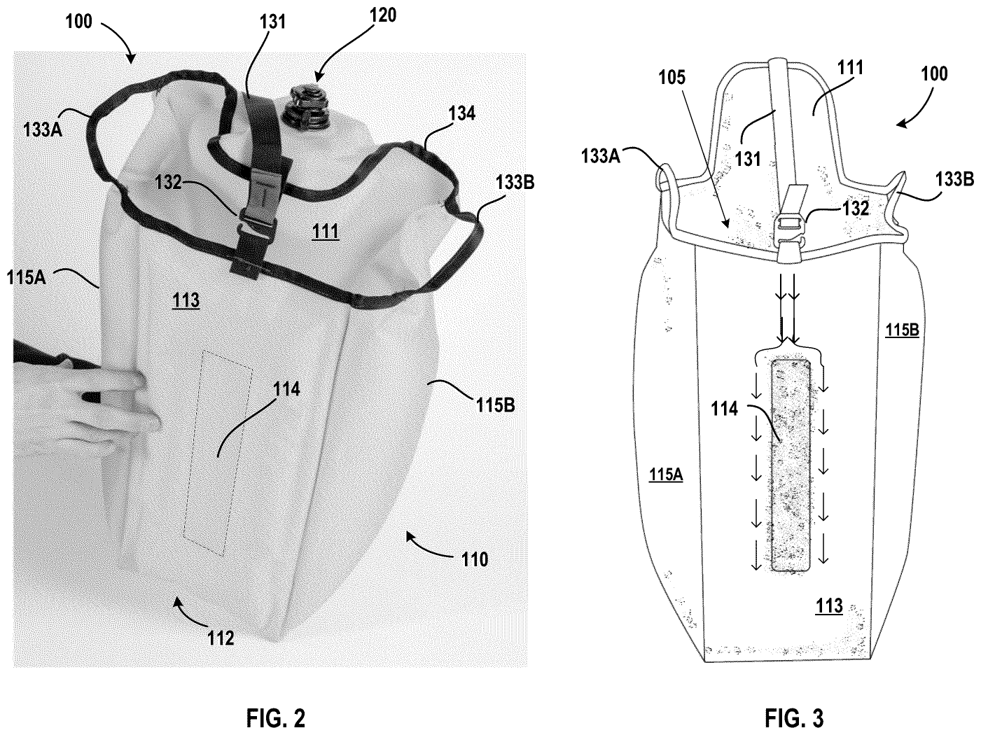

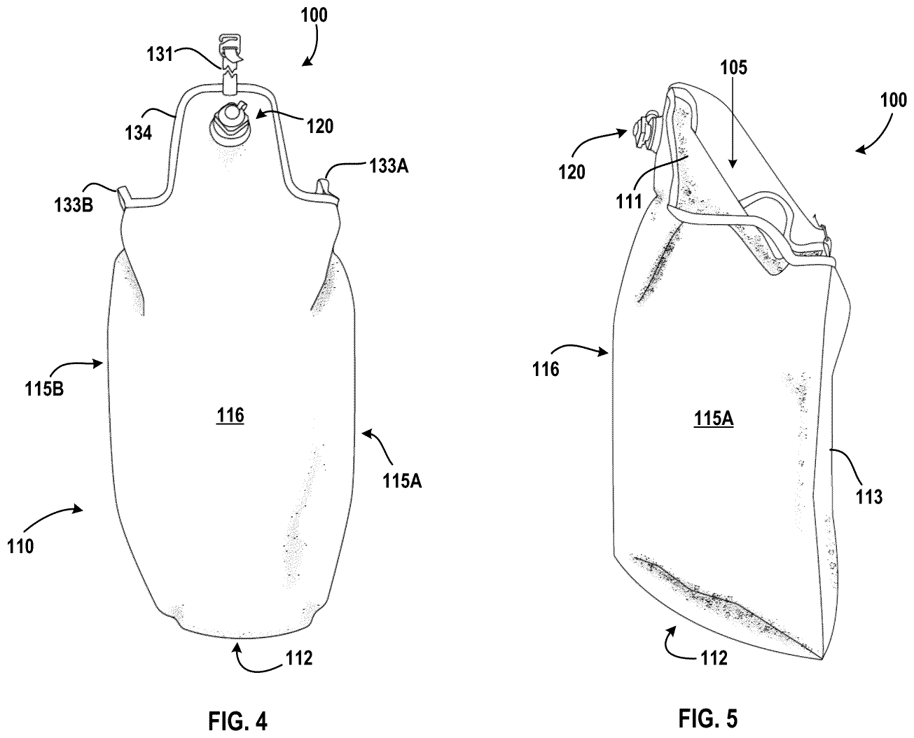

Now turning to the figures, FIGS. 2 to 5 should be referred to for the following discussion; these figures show perspective, front, rear and left-side views of the example inflatable bag 100. Inflatable bag 100 has a flexible body which may include a series of panels or surfaces. For example, body 110 includes a flexible, closable top panel or flap 111. Top flap 111 can be opened by removing a fastening strap 131 out of its corresponding slotted buckle 132. With the fastening strap 131 loosened, the top flap 111 can be opened to expose the interior cavity 105 of the inflatable bag 100.

Body 110 may include additional material panels or surfaces; as shown this may include a bottom panel or surface 112, a front panel or surface 113 which includes a centrally located baffle region or baffle 114 formed thereon. The purpose of this baffle 114 is to provide an ergonomic improvement related to how the backpack 50 with the inflated bag 110 therein rests against the back of the wearer, as opposed to no baffle 114. The use of the baffle 114 in the front panel 113 enables a “flattening” of the front panel 113 as it faces the back of the wearer of the backpack 50. Without the baffle 114, the front panel 113, during an inflation evolution to inflate the body 110 (and hence bag 100) would create a non-flat, balloon shape or bulge in the front panel 113 (as this exterior surface expands outward with the incoming pressurized air). This balloon shape or bulge would push against the back of the wearer, creating discomfort and some instability.

As best shown in FIG. 3, since the baffle 114 is centrally located in the front panel 113 as a region where air will not inflate, the airflow of the air supply for inflation in the front panel 113 of the body 110 will create two cylinder-like paths (either side of baffle 114) down the front panel 113 and hence against the wearer's back. This will enable a generally flatter profile for the front panel 113 of the bag 100 so as to relieve pressure on the spine of the wearer, when the inflated bag 100 is installed fully inflated within the primary storage compartment of the backpack 50. The baffle 114 therefore provides an ergonomic enhancement in flattening the profile of the front panel 113 during inflation of the inflatable bag 100.

Body 110 additionally includes a rear panel or surface 116 which includes an inflation valve unit 120 sewn therein at an upper end thereof, and side panels or surfaces 115A and 115B, each having a corresponding webbing strap portion 133A and 133B arranged at upper ends of the respective side panel 115A and 115B. Webbing strap portions 133A and 133B provide a handle function for facilitating removal of the inflatable bag 100 from a primary storage compartment (not shown) of the backpack 50.

The webbing 134 that makes up the webbing strap portions 133A and 133B is shown as to be continuous and is sewn or otherwise attached around the upper end of the body 110 and top flap 111, as best shown in FIG. 3. Webbing 134 may be embodied as 1″ nylon webbing stitched with a suitable commercial-grade thread (such as tex 70 and/or tex 90 bonded polyester thread, as one example).

The entire body 110, inclusive of the aforementioned panels or surfaces 111, 112, 113, 114, 115A/115B and 116 can be made out of a suitable resilient plastic material. One example may be a thermoplastic polyurethane (TPU) coated fabric, where the base fabric may be, as merely exemplary, any of a nylon fabric, polyester fabric, ultra-high molecular weight (UHMW) polyethylene fabric, combinations thereof, and the like. A TPU coated fabric uses polymer blending to create a uniquely strong TPU coating, which is then applied to a variety of high-quality base fabrics such as noted above using hot melt or spread coating on complementary production lines. The resultant textiles offer excellent resistance to extreme weather conditions, chemicals, abrasion and punctures, as well as high UV stability. Waterproof TPU fabrics bond a thermoplastic polyurethane membrane to a base textile, such as nylon or polyester fabric, to create a wide range of high-performing materials that maintain their flexibility yet are durable and waterproof. For example, a TPU coated UHMW fabric is an extremely tough plastic with high abrasion and wear resistance. The versatility of polyethylene makes UHMW fabrics popular for countless industrial applications that require durability, low friction, and chemical resistance. Although not limiting to the present example embodiment, the inventors have investigated commercially viable textiles from the above TPU-coated base fabrics for body 110, including textile products having a 1680 denier 6 mil TPU coating, and a 200 denier 2 mil TPU coating.

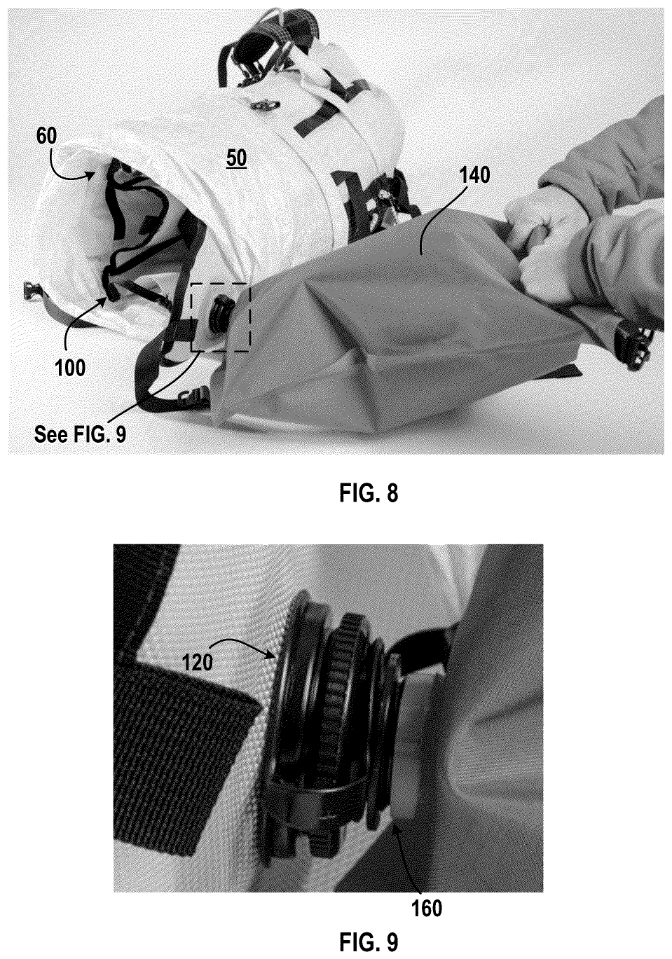

The inflatable valve unit 120, which is manufactured into the body 110 or otherwise sewn therein, is located at the upper end of the body 110 on the rear surface 116. As to be shown in more detail later with regard to FIGS. 8 to 10, the inflation valve unit 120 includes a “female” inflation valve that, in one example, may be embodied as a one-way TPU Boston valve. Anyone who has used or owns an inflatable boat or raft is familiar with Boston valves, which are designed to let in the air when pumping (inflating) while shielding the boat (or here inflatable bag 100) from air seepages. A wearer can directly blow into the female inflation valve of valve unit 120 to inflate bag 100. Alternatively, the female inflation valve of valve unit 120 is designed to cooperate with a male air supply valve from a separate air supply source, such as a bag pump or other powered air pump.

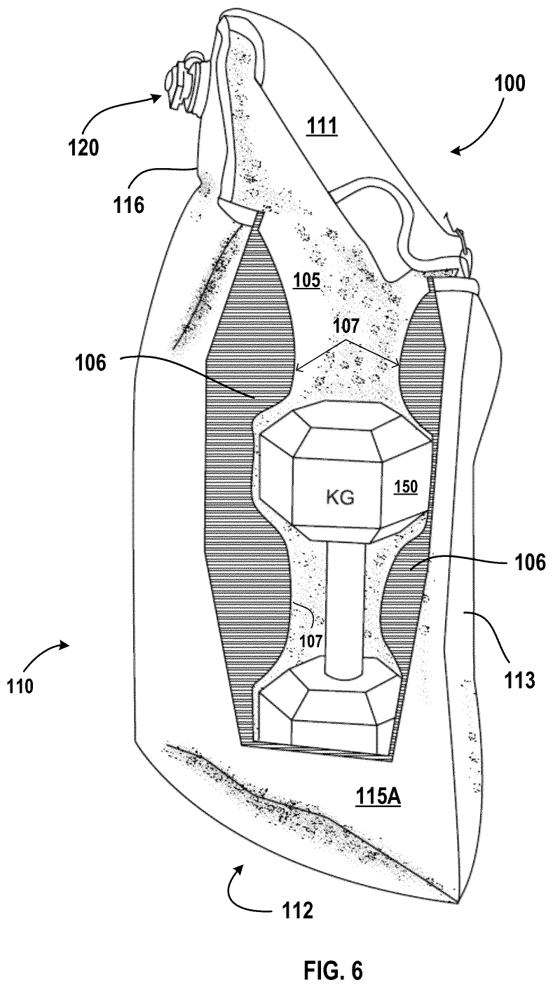

FIG. 6 is a partial cutaway rendering of the left-side elevational view previously shown in FIG. 5. The cutaway shown in left side panel 115A is provided to give the reader a look-in view of what the interior of the body 110 would look like around a loose weight. Here in FIG. 6, the body 110 or bag 100 is fully inflated and a loose weight 150 (shown here as a dumbbell) has been placed into the cavity 105 of the body 110 with its front flap 110 open. The weight 150 deforms the interior inflated portion 106 of the body 110 at various contact points on the inner facing surface 107 of the cavity, around the entire inner circumference of cavity 105. Thus, weight 150 is firmly secured within the inflated bag 110, there will be no loose movement on a hike or walk with this inflated back 100 placed within backpack 50 and the fastening strap 131 in slotted buckle 132 tightened to close the top flap 111.

Thus, FIG. 6 is provided to show that after the bag has been inflated via the inflation valve unit receiving pressurized air therethrough, the surfaces 107 of the interior cavity 105 expand (during inflation) inward and external surfaces of the body 110 (i.e., the various panels or surfaces 1111, 112, 113, 155A/B, 116 expand (during inflation) outward to fill the volume of the internal storage compartment (not shown) of the backpack 50. Thus, any weighted items (such as weight 150) added into the interior cavity 105 in the body 110 with bag 100 inflated are firmly secured, as the surfaces 107 of the inflated interior portions 106 in the cavity 105 bear against the weighted items, eliminating any shifting of the weighted items during a wearer's movement activities with a weighted backpack with the inflated bag 100 filling the volume of the interior storage compartment.

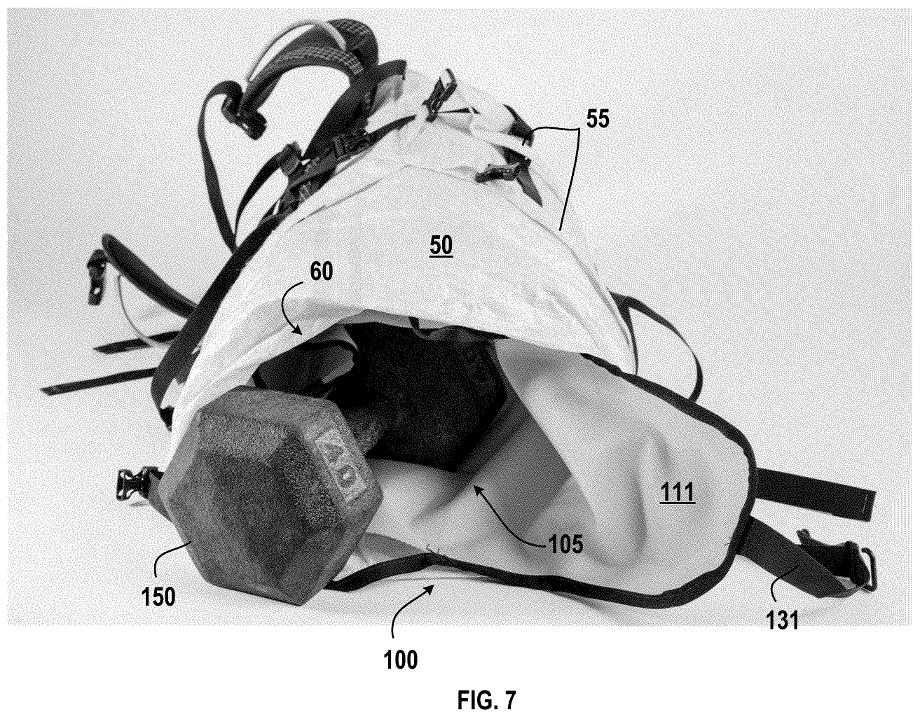

FIG. 7 is a photograph of the backpack 50 with its top opened and laying on its side, the inflatable bag 100 having been inserted therein, with a weight 150 shown for insertion into the cavity 105 of the inflatable bag 100. Here, the inflatable bag 100 has been fully inflated in advance and already inserted into the primary interior storage compartment 60 of the backpack 50. With the fastening strap 131 removed out of its slotted buckle 132 and the top flap 111 opened to expose the cavity 105 of the inflatable bag 100, any kind of weight 150 (here shown as a 40-pound dumbbell), can be inserted into the cavity 105.

FIG. 8 is a photograph of the inflatable bag with weight shown in FIG. 7 therein, to illustrate how an inflation valve unit of the inflatable bag connects to and cooperates with a corresponding air valve in a storage bag which is used as an air pump for inflation. FIG. 9 is an enlarged view of the boxed area in FIG. 8 to show more detail around the valve connections between the inflatable bag and its storage. Recall previously that the inflated bag 100 can also be hooked up to an external air supply, such as a pump bag or a portable self-powered air pump.

Recall previously that it was noted where the inflatable bag 100, when not in use can be rolled up and stored within a companion storage bag. This storage bag 140 is shown in FIGS. 8 and 9 and it has another function; it serves as an air pump (bag pump). Namely, storage bag 140 also has an air supply valve that can attached to the inflatable so that the storage bag can be used as an air pump.

In FIGS. 8 and 9, the storage bag 140 has an air supply valve assembly 160 that is sewn, manufactured or otherwise formed therein. This air supply valve assembly 160 serves as the “male” valve that can mate up, via conventional friction fit, with the female inflation valve in inflation valve unit 120. Once connected (friction fit between the 2 valves), the wearer starts rolling up the storage bag 140 (see FIG. 8) from the bottom end toward the top. This creates a pressurized air pocket inside storage bag 140 which serves as a pressurized air source that is ported into the body 110 of the bag 100 via the connected valve unit 120/valve assembly 160. Thus, the storage bag 140 can be used as an air pump; known as a bag pump in the industry.

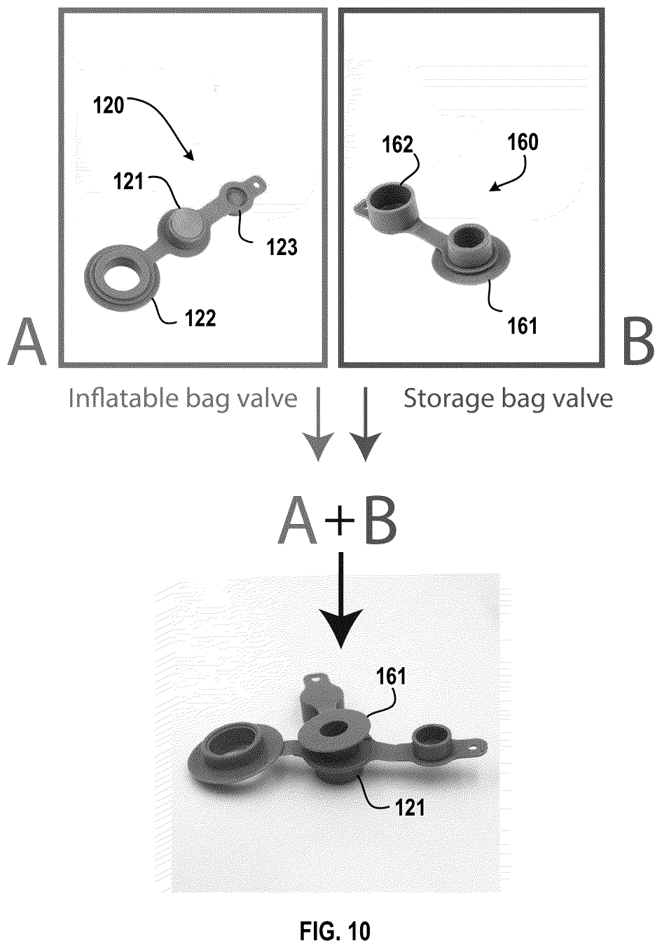

FIG. 10 is a diagram showing the above constructions of the valve unit 120 and valve assembly 160 to connect the inflatable bag 100 to the storage bag 140 serving as a bag pump. In this example, there overall inflation assemblage is composed of two separate components, one on the bag 100 side, one on the storage bag 140 side. Here, the first component is the inflation valve unit 120 which is manufactured into the inflatable bag 100 so as to be accessed on the outside upper rear surface 116 of the inflatable bag 100 as shown. The second component is the air supply valve assembly 160, which is an air supply valve sewn into the roll-up bag pump (storage bag 140). The valve constructions in FIG. 10 are exemplary off the shelf components, and cooperate via a friction fit. Box A shows the inflation valve unit 120, which in an example includes the main female inflation valve 121 (one way) with a flexibly attached bezel 122 and closer 123. Box B is the air supply valve assembly 160 for the storage bag 140 in its pump bag functionality. The air supply valve assembly 160 includes the male air supply valve 161 and flexibly attached cap 162, with engagement of the male air supply valve 161 into female inflation valve 121 (bezel 122 removed solely to see the connection better) shown in the A+B combination in FIG. 10. The connected state between bags 100/140 is best seen in FIG. 9. Of course, without storage bag 140 serving as a bag pump, the inflatable bag 100 can also be inflated with the wearer's mouth providing air supply, blowing directly into the female inflation valve 121.

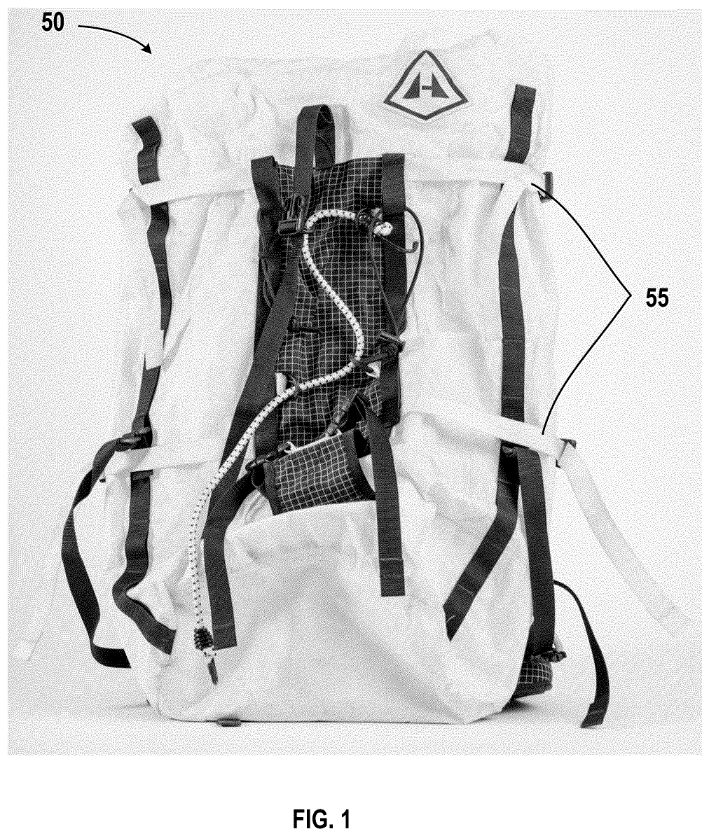

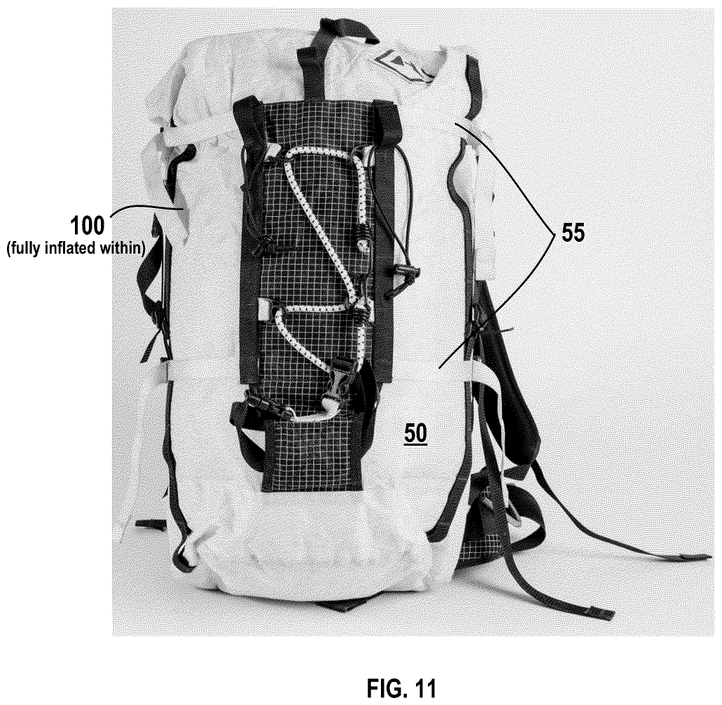

FIG. 11 is a rear plan view of the conventional backpack shown in FIGS. 1, 7 and 8 with the fully inflated bag 100 provided therein. This is how backpack 50 would look with the fully inflated bag 100 therein and the compression straps 55 tightened up. There is no movement of any loose weight 150 therein as any weight 150 is firmly secured within, as best shown in FIG. 6.

Accordingly, within the backpack 50's interior storage compartment (not shown in FIG. 11) the internal surfaces within the interior cavity 105 of bag 100 (FIG. 6) are inflated inward, and external surfaces of the body 110 of the inflated bag 100 are inflated outward (with the exception of the baffle 114 to provide a generally flat profile against the wearer's back) to fill the volume of the internal storage compartment inside backpack 50. Thus, any weighted items (such as weight 150) added into the interior cavity 105 of the inflated bag 100 are firmly secured as the surfaces 107 of the inflated portions 106 (see again FIG. 6 inside of the interior cavity 105) bear against the weighted items. This desirably eliminates any shifting of the weighted items during a wearer's movement activities with the weighted backpack 50, since the inflated bag 100 fills the volume of the backpack 50's interior storage compartment. The backpack 50 shown in FIG. 11 thus feels full and stable on the back of the wearer, and the loading of the weight therein is distributed to generate desirable force vectors toward the back of the wearer during movement activities.

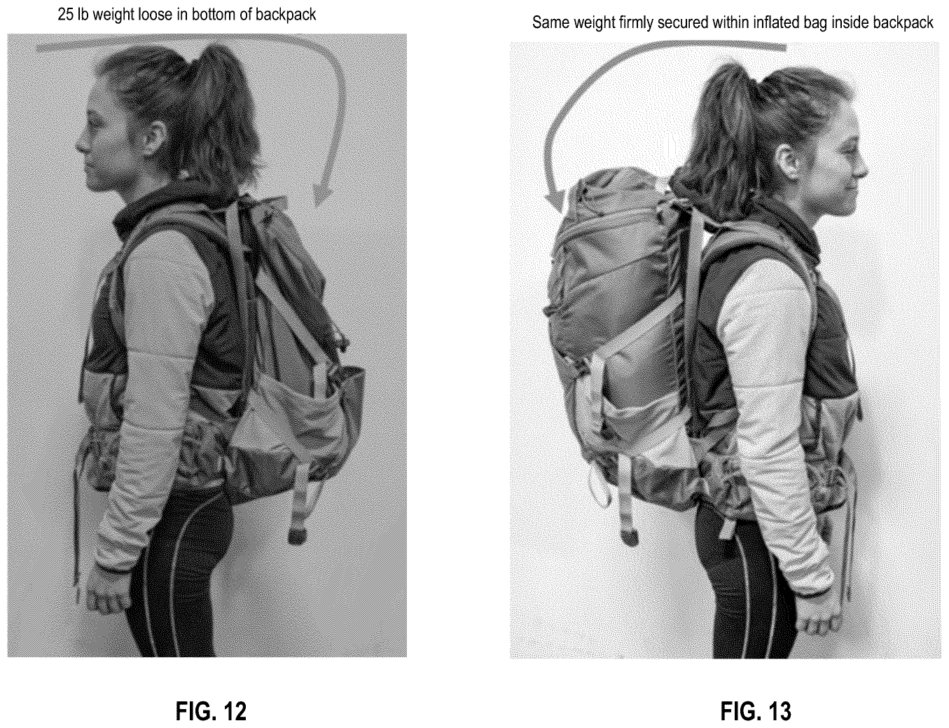

FIG. 12 is a photographed side view of a person wearing a backpack with a loose 25-pound weight inserted into the bottom of the backpack's primary storage compartment. This is for comparison to FIG. 13, which is the same person, but with the same weight firmly secured within the fully inflated bag 100 inside the backpack 50's primary storage compartment 60, according to the example embodiment. The chance of injury on a difficult movement activating with the backpack is enhanced in the configuration of FIG. 12, but is substantially lessened with the backpack 50 volume filled with the inflated bag 100 containing weight therein. The backpack in FIG. 12 is loose, and the weight therein can move substantially; this is not the case with the backpack configuration of FIG. 13.

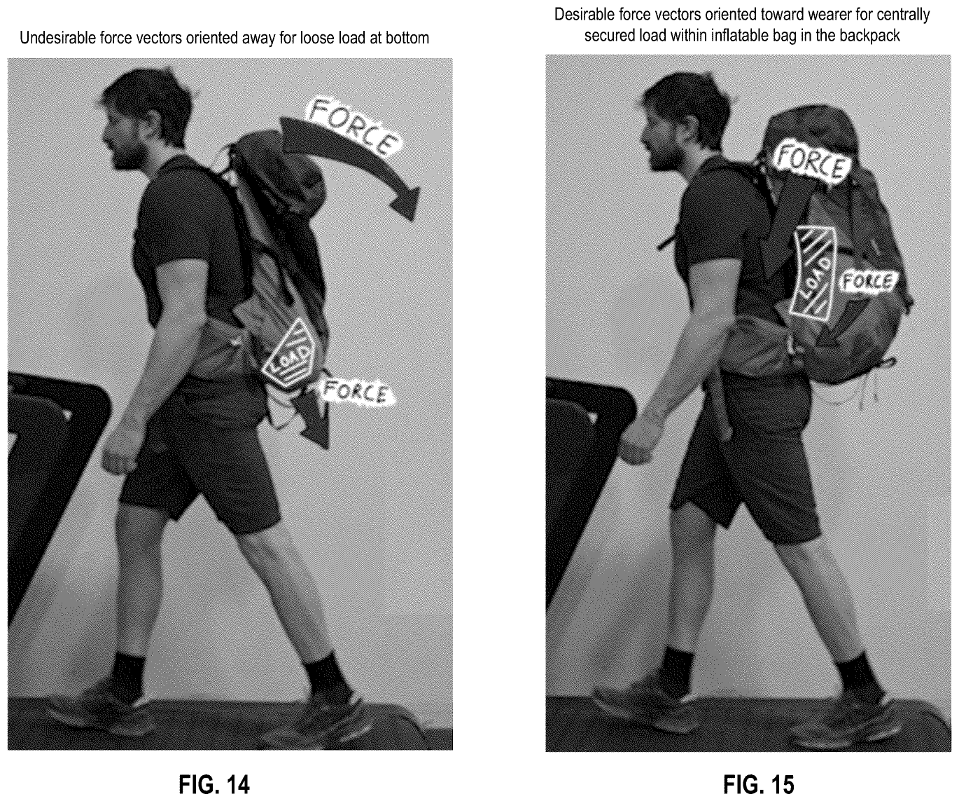

FIG. 14 is a photographed side view of a person wearing a backpack and walking on a treadmill with a loose weight within the backpack's primary storage compartment, so as to show load location of the loose weight and force vectors imparted away from the person. The load location is undesirable in the backpack of FIG. 14, and can cause discomfort should the load move back and forth along the lower back of the wearer of the backpack. The force vectors created by the walking/hiking movement with the wearer's wearing backpack on the treadmill are undesirable, as they are generated away from the wearer due to the instability imparted by, and the location of the loose weight in the backpack. Compare this to the same treadmill walking situation shown in FIG. 15. Here, the same person is shown walking on the treadmill. Note the different load location and desirable force vectors imparted towards the wearer. These are generated because the same weight is now firmly secured within the fully inflated bag inside (not shown), which fills the volume inside the backpack's primary storage compartment. In both FIGS. 13 and 15, even should the wearer shake the completely full backpack with the fully inflated bag 100 therein, nothing within the interior cavity 105 of the body 110 of bag 100 moves (see FIG. 6); it feels to the wearer like a completely full and evenly weight-distributed backpack.

The example embodiment being thus described, it will be obvious that the same may be varied in many ways. Such variations are not to be regarded as departure from the example embodiment, and all such modifications as would be obvious to one skilled in the art are intended to be included herein.

What is claimed:

1. A removable, inflatable bag for insertion into an interior storage compartment of a backpack, comprising:
a body having an interior cavity for storing one or more loose, weighted items through a re-closeable opening at a top end thereof, and
an inflation valve unit arranged on an external surface of the body and adapted to receive pressurized air therethrough to fully inflate the body, such that internal surfaces within the interior cavity of the body expand inward, and external surfaces of the body expand outward to fill the volume of the internal storage compartment of the backpack, wherein
with the inflatable bag arranged in the interior storage compartment of the backpack and fully inflated, and with the one or more loose, weighted items contained within the interior cavity of the body and the top end opening of the fully inflated bag closed, the surfaces of the interior cavity bear against the one or more loose, weighted items to prevent any movement thereof, eliminating any shifting of the one or more loose, weighted items during a wearer's movement activities with the backpack.

2. The removable, inflatable bag of claim 1, wherein the body includes a fastening strap with slotted buckle attached at a top end thereof and a flexible top flap that allows access through the re-closeable opening into the interior cavity of the body when the fastening strap is loosened or otherwise removed from the slotted buckle, the fastening strap secured into the slotted buckle and tightening to maintain the top flap closed over the re-closeable opening.

3. The removable, inflatable bag of claim 1, wherein the body of the inflatable bag includes a front panel adapted to rest against the back of the wearer when the inflatable bag is inflated and arranged within the backpack, the front panel including a centrally located baffle that serves as a region in the front panel that will not inflate as the body of the bag is being inflated, the incoming air for inflation forming two inflated cylinder-like inflated portions along either side of the front panel that enable a generally flatter profile for the front panel of the body as it rests against the back of the wearer inside the backpack, so as to relieve pressure on the spine of the wearer.

4. The removable, inflatable bag of claim 1, wherein the body includes a rear panel, the inflation valve unit attached to the body at an upper portion of the rear panel.

5. The removable, inflatable bag of claim 1, wherein the inflatable bag when not in use is storable in a storage bag that serves a dual function as a bag pump so as to provide pressurized air to inflate the inflatable bag.

6. The removable, inflatable bag of claim 1, wherein the inflation valve unit includes a female inflation valve that is configured to mate via a friction fit connection to a male air supply valve arranged on a separate storage bag that is useable as a bag pump by rolling up the storage bag from the bottom to the top to create a pressurized air pocket inside that storage bag that serves as the supply air source to inflate the body of the inflatable bag, the inflatable bag when not in use being storable in the storage bag.

7. The removable, inflatable bag of claim 1, wherein, as the wearer is moving with the backpack filled with the inflatable bag fully inflated and firmly securing weighted items therein, force vectors generated during movement are desirably oriented toward the wearer's back and the load of the weighted items is distributed, as opposed to a backpack worn by the wearer and containing only loose weighted items therein that drop to the bottom of the interior storage compartment so that force vectors generated by the wearer wearing the backpack during movement are undesirably oriented away from the back of the wearer.

8. A combination, comprising:
a backpack having a re-closable opening at a top end thereof and an interior storage compartment having a volume that is accessible through the re-closable opening, and
an inflatable bag adapted for insertion into the interior storage compartment of the backpack via the re-closable opening of the backpack for use in weighted backpack training activities by a wearer of the backpack, the inflatable bag further comprising:
a body having an interior cavity for storing weighted items therein through a re-closeable opening at a top end thereof, and
an inflation valve unit adapted arranged on a surface of the body and adapted to receive pressurized air therethrough to fully inflate the body of the inflatable bag, such that internal surfaces within the interior cavity of the body expand inward, and external surfaces of the body expand outward to fill the volume of the internal storage compartment of the backpack, wherein
any weighted items added into the interior cavity of the body with the inflatable bag fully inflated are firmly secured as the surfaces of the interior cavity bear against the weighted items, eliminating any shifting of the weighted items during a wearer's movement activities wearing the weighted backpack, the inflated bag therein filling the volume of the interior storage compartment.

9. The combination of claim 8, wherein the body of the inflatable bag includes a front panel adapted to rest against the back of the wearer when the inflatable bag is inflated and arranged within the backpack, the front panel including a centrally located baffle that serves as a region in the front panel that will not inflate as the body of the bag is being inflated, the incoming pressurized air for inflation forming two inflated cylinder-like inflated portions along either side of the centrally located baffle in the front panel that enable a generally flatter profile for the front panel of the body as it rests against the back of the wearer inside the backpack, so as to relieve pressure on the spine of the wearer.

10. The combination of claim 8, wherein the inflation valve unit includes a female inflation valve that is configured to mate via a friction fit connection to a male air supply valve arranged on a separate storage bag that is useable as a bag pump by rolling up the storage bag from the bottom to the top to create a pressurized air pocket inside that storage bag that serves as the supply air source to inflate the body of the inflatable bag through the friction fit connection into the body thereof, the inflatable bag when not in use being storable in the storage bag.

11. The combination of claim 8, wherein, as the wearer is moving with the backpack filled with the inflatable bag fully inflated and firmly securing weighted items therein, force vectors generated during movement are desirably toward the wearer's back and the load of the weighted items is distributed, as opposed to a backpack worn by the wearer and containing only loose weighted items therein that drop to the bottom of the interior storage compartment so that force vectors generated by the wearer wearing the backpack during movement are undesirably oriented away from the back of the wearer.

12. A combination, comprising:
an inflatable bag adapted for insertion into an interior storage compartment of a backpack via a re-closable opening in the backpack for use in weighted backpack training activities by a wearer of the backpack, the inflatable bag having an interior cavity for storing weighted items therein, and having an inflation valve unit arranged thereon that is adapted to receive pressurized air therein, and
a storage bag for storing the inflatable bag therein when not in use within the backpack, the storage bag serving a dual function as a bag pump to provide pressurized air into the inflation valve unit to fully inflate the inflatable bag, wherein
for any weighted items added into an interior cavity of the inflatable bag when the bag has been fully inflated by the storage bag, the fully inflated bag arranged within the interior storage compartment of the backpack to realize a weighted backpack, the weighted items are firmly secured as inflated surfaces within the interior cavity bear against the weighted items, eliminating any shifting of the weighted items during a wearer's movement activities with the weighted backpack, the inflatable bag fully inflated filling the volume of the interior storage compartment.

13. The combination of claim 12, wherein as the inflation valve unit receives pressurized air therethrough from the storage bag to fully inflate the inflatable bag, internal surfaces within the interior cavity of the inflatable bag expand inward, and external surfaces of the body expand outward to fill the volume of the internal storage compartment of the backpack.

14. The combination of claim 12, wherein the inflation valve unit includes a female inflation valve that is configured to mate via a friction fit connection to a male air supply valve arranged on the storage bag in order to fully inflate the inflatable bag.

15. The combination of claim 14, wherein the storage bag is useable as a bag pump by rolling up the storage bag from its bottom to top to create a pressurized air pocket inside the storage bag that serves as the supply air source to inflate the inflatable bag through the friction fit connection.

16. The combination of claim 12, wherein the inflatable bag includes a front panel adapted to rest against the back of the wearer when the inflatable bag is inflated and arranged within the interior compartment of the backpack, the front panel including a centrally located baffle that serves as a region in the front panel that will not inflate as inflatable bag is being inflated, the baffle serving as an ergonomic enhancement in flattening the profile of the front panel during inflation of the inflatable bag.

17. The combination of claim 16, wherein the incoming pressurized air from the storage bag for inflation forms two inflated cylinder-like inflated portions along either side of the centrally located baffle in the front panel that enable a generally flatter profile for the front panel of the body as it rests against the back of the wearer inside the backpack, so as to relieve pressure on the spine of the wearer.
